(19) United States
(12) Patent Application Publication
(10) Pub. No.: US 2005/0228377 A1
(43) Pub. Date: Oct. 13, 2005

(54) SPINAL CROSS-CONNECTORS

(75) Inventors: Nam T. Chao, Marlborough, MA (US); Chris Rybicki, Stamford, CT (US); Bryan S. Jones, Norwood, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: DEPUY SPINE, INC., Raynham, MA (US)

(21) Appl. No.: 10/709,019

(22) Filed: Apr. 7, 2004

(51) Int. Cl. A61B 17/58

(52) U.S. Cl. 606/61

(57) ABSTRACT

An implantable spinal cross-connector is provided for connecting one or more spinal fixation devices, and more preferably for connecting two spinal fixation rods that are implanted within a patient's spinal system. In general, the cross-connector includes a central portion having at least one connector member formed on a terminal end thereof and having first and second opposed jaws, at least one of which is selectively movable between a first, open position wherein the first and second jaws are positioned a distance apart from one another, and a second, closed position, wherein the first and second jaws are adapted to engage a spinal fixation element therebetween. The cross-connector also includes a locking mechanism having a shank that is receivable within a non-expandable bore formed in the connector member. The locking mechanism is adapted to come into contact with each of the first and second jaws to selectively lock the first and second jaws in a fixed position with respect to one another.

### FIELD OF THE INVENTION

The present invention relates to spinal fixation devices, and in particular to a cross-connector for connecting spinal fixation devices, such as spinal fixation rods implanted in a patient's spinal system.

### BACKGROUND OF THE INVENTION

Spinal fixation devices are used in orthopedic surgery to align and/or fix a desired relationship between adjacent vertebral bodies. Such devices typically include a spinal fixation element, such as a relatively rigid fixation rod, that is coupled to adjacent vertebrae by attaching the element to various anchoring devices, such as hooks, bolts, wires, or screws. Often two rods are disposed on opposite sides of the spinous process in a substantially parallel relationship. The fixation rods can have a predetermined contour that has been designed according to the properties of the target implantation site, and once installed, the rods hold the vertebrae in a desired spatial relationship, either until desired healing or spinal fusion has taken place, or for some longer period of time.

Spinal cross-connectors are often used in conjunction with spinal fixation devices to provide additional stability to the devices. For example, it has been found that when a pair of spinal rods are fastened in parallel on either side of the spinous process, the assembly can be significantly strengthened by using a cross-connector to bridge the pair of spinal rods. The connectors are typically in the form of a rod having a clamp formed on each end thereof for mating with a spinal rod.

While current spinal cross-connectors have proven effective, difficulties have been encountered in mounting the cross-connectors, and maintaining them in a desired position and orientation with respect to the spinal rod, or other spinal fixation device to which they are attached. In particular, the clamp assemblies often consist of several parts which increase the manufacturing costs and make surgical application tedious. Since the cross-connector is often applied as the last step in a lengthy surgical procedure, ease of application is paramount. Fixation of the cross-connector to spinal rods can also be difficult where the rods are not parallel to one another, or they are diverging/converging with respect to one another.

Accordingly, there presently exists a need for an improved spinal cross-connector that can be easily installed and that securely mates to and connects spinal fixation devices.

### BRIEF SUMMARY OF THE INVENTION

The present invention provides an implantable spinal cross-connector for connecting spinal fixation devices, and more preferably for connecting two spinal fixation rods to one another. In one embodiment, the cross-connector includes a central portion having at least one connector member formed on a terminal end thereof and having first and second opposed jaws, at least one of which is selectively movable between a first, open position wherein the first and second jaws are positioned a distance apart from one another, and a second, closed position wherein the first and second jaws are adapted to engage a spinal fixation element therebetween. The cross-connector also includes a locking mechanism having a shank that is receivable within a bore formed in the connector member. The locking mechanism is adapted to come into contact with each of the first and second jaws to selectively lock the first and second jaws in a fixed position with respect to one another.

The connector member(s) can have a variety of configurations, and a variety of techniques can be used to allow the jaws to move between the open and closed positions. In one embodiment, the first jaw can be integrally formed with the connector, and the second jaw can be independent from and pivotally mated to the first jaw. A pivot pin preferably extends through the first and second jaws to allow pivotal movement of the second jaw with respect to the first jaw. The connector member can also include a non-expandable bore that is formed in the first jaw and that includes an enlarged opening formed therein for seating a head formed on the shank of the locking mechanism. The enlarged opening is preferably formed adjacent to the second jaw such that the head of the locking mechanism is effective to pivot the second jaw into the second, closed position when the head is disposed within the enlarged opening. In an exemplary embodiment, the head of the locking mechanism is non-eccentric.

In another embodiment, the first and second jaws can include a slot formed therebetween that is adapted to allow movement of the first and second jaws between the first, open position and the second, closed position. Preferably, a non-expandable bore extends through the first and second jaws across the slot such that the locking mechanism is effective to close the slot when the locking mechanism is advanced into the non-expandable bore, thereby moving the first and second jaws from the first, open position to the second, closed position. In an exemplary embodiment, the non-expandable bore includes a non-threaded portion that is formed in the first jaw and a threaded portion that is formed in the second jaw, and the shank of the locking mechanism includes a non-threaded proximal portion that is adapted to sit within the non-threaded portion of the non-expandable bore formed in the first jaw, and a threaded distal portion that is effective to mate with the threaded portion of the non-expandable bore formed in the second jaw. A head is preferably formed on the non-threaded proximal portion of the shank of the locking mechanism that is receivable within an enlarged opening of the non-expandable bore formed in the first jaw. In use, the locking mechanism is effective to pull the first and second jaws toward one another, or it can pull the second jaw toward the first jaw, into the second, closed position when the locking mechanism is advanced into the non-expandable bore.

The central portion of the connector member can also have a variety of configurations, and in one embodiment it can be a substantially elongate member having an adjustable length, and more preferably it can be formed from first and second transverse members that are slidably matable to one another. The first transverse member can include a female mating element, and the second transverse member can include a male mating element that is adapted to be received by the female mating element. A central locking mechanism can be provided for locking the first and second transverse members at a fixed position with respect to one another. In a further embodiment, the first and second transverse members can be angularly adjustable with respect to one another along a longitudinal axis of the spinal cross-connector.

The present invention also provides a spinal rod and connector system that includes at least one spinal rod, and a spinal cross-connector. The cross-connector has at least one connector member formed thereon and including first and second opposed jaws that are movable between an open position and a closed position in which the jaws are adapted to engage said spinal rod, and a locking mechanism having a head and a shank that are receivable with a bore having a proximal, head-receiving portion, and a distal, shank-engaging portion that is formed in the first jaw at a distance apart from the second jaw. The distal, shank-engaging portion of the bore preferably has a uniform diameter along a length thereof. In use, the locking mechanism is adapted to lock the first and second jaws in the closed position.

### BRIEF DESCRIPTION OF THE DRAWINGS

The invention will be more fully understood from the following detailed description taken in conjunction with the accompanying drawings, in which:

FIG. 1A is a side perspective view of one embodiment of a spinal cross-connector according to the present invention;

FIG. 1B is a disassembled view of the spinal cross-connector shown in FIG. 1A;

FIG. 2A is a cross-sectional view of a central portion of the spinal cross-connector shown in FIG. 1A;

FIG. 2B is a disassembled, perspective view of a central locking mechanism on the spinal cross-connector shown in FIGS. 1A;

FIG. 3A is a side perspective view of one of the connector members on the spinal cross-connector shown in FIG. 1A;

FIG. 3B is a cross-sectional view of the connector member shown in FIG. 3A;

FIG. 4A is a side perspective view of another embodiment of a spinal cross-connector according to the present invention;

FIG. 4B is a disassembled view of the spinal cross-connector shown in FIG. 4A;

FIG. 5A is a cross-sectional view of a central portion of the spinal cross-connector shown in FIG. 4A;

FIG. 5B is a disassembled, perspective view of a central locking mechanism on the spinal cross-connector shown in FIGS. 4A;

FIG. 6A is a side perspective view of one of the connector members on the spinal cross-connector shown in FIG. 4A;

FIG. 6B is a cross-sectional view of the connector member shown in FIG. 6A;

FIG. 7 is a side perspective view of yet another embodiment of a spinal cross-connector in accordance with the present invention;

FIG. 8A is a perspective view of spinal cross-connector mated to two spinal fixation rods in accordance with yet another embodiment of the present invention;

FIG. 8B is a disassembled view of a portion of the spinal cross-connector shown in FIG. 8A; and

FIG. 9 is a disassembled view of another embodiment of a spinal cross-connector for mating to two spinal fixation rods.

### DETAILED DESCRIPTION OF THE INVENTION

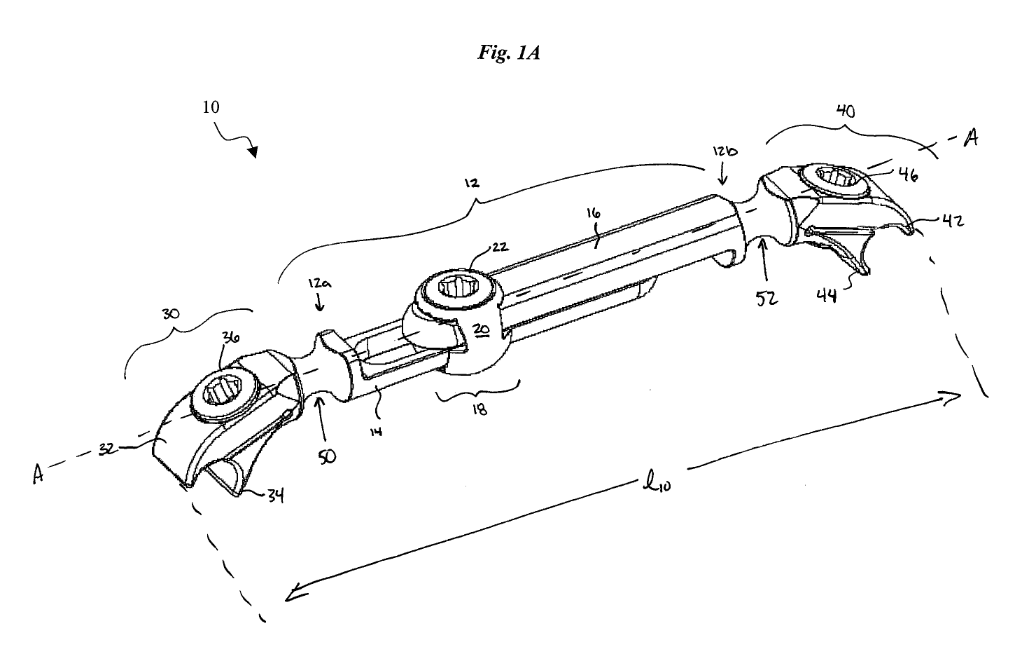

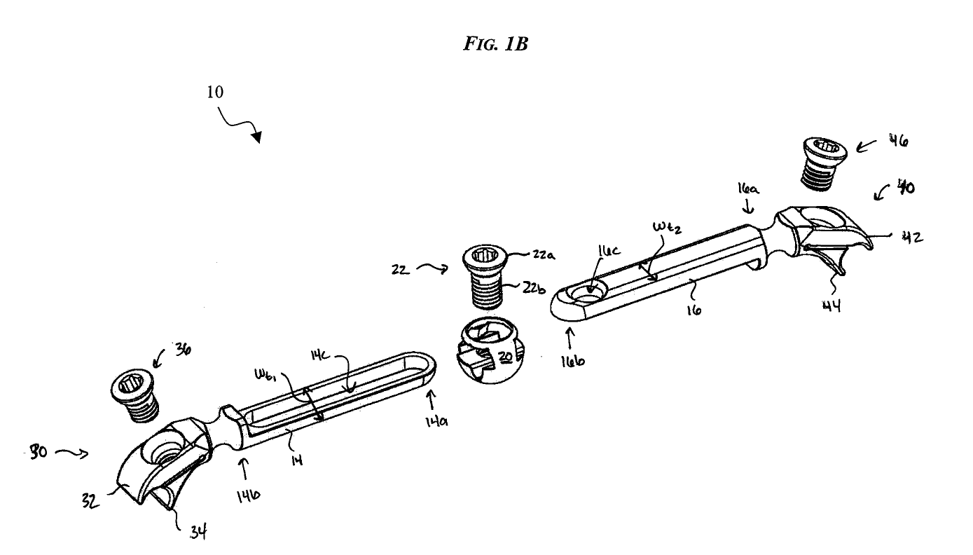

The present invention provides a spinal cross-connector for connecting one or more spinal fixation devices, and more preferably for connecting two spinal fixation rods that are implanted within a patient's spinal system. FIGS. 1A-9 illustrate various embodiments of exemplary cross-connectors 10, 100, 200, 300, 400 in accordance with the present invention, and in each of the illustrated embodiments the cross-connector 10, 100, 200, 300, 400 generally includes a central portion 12, 112, 212, 312, 412 having at least one connector member, e.g., connector members 30, 40, 130, 140, 230, 240, 330, 340, 430, 440 formed on a terminal end thereof. Each connector member 30, 40, 130, 140, 230, 240, 330, 340, 430, 440 can have a variety of configurations. In the embodiments illustrated in FIGS. 1A-7, the connector members 30, 40, 130, 140, 230, 240 each have first and second opposed jaws 32, 34, 42, 44, 132, 134, 142, 144, 232, 234, 242, 244, at least one of which is selectively movable between a first, open position wherein the first and second jaws 32, 34, 42, 44, 132, 134, 142, 144, 232, 234, 242, 244 are positioned a distance apart from one another, and a second, closed position wherein the first and second jaws 32, 34, 42, 44, 132, 134, 142, 144, 232, 234, 242, 244 are adapted to engage a spinal fixation element therebetween. Each connector member 30, 40, 130, 140, 230, 240, 330, 340, 430, 440 can also include a locking mechanism 36, 46, 136, 146, 236, 246, 336, 346, 436, 446 that is adapted to selectively lock a spinal fixation element to the connector member 30, 40, 130, 140, 230, 240, 330, 340, 430, 440. In the embodiments illustrated in FIGS. 1A-7, the locking mechanism 36, 46, 136, 146, 236, 246 is effective to lock the first and second jaws 32, 34, 42, 44, 132, 134, 142, 144, 232, 234 in a fixed position with respect to one another such that the jaws 32, 34, 42, 44, 132, 134, 142, 144, 232, 234 can engage a spinal fixation element disposed therebetween.

A person skilled in the art will appreciate that while each cross-connector 10, 100, 200, 300, 400 is described herein as being adapted to engage a spinal fixation element, and in particular a spinal fixation rod, that a cross-connector of the present invention can be configured to engage a variety of spinal fixation devices, such as anchors, cables, fixation plates, etc. Moreover, the cross-connector can include only one connector member for engaging a spinal fixation device, and the opposed terminal end of the cross-connector can be adapted for other uses. For example, the opposed terminal end of the cross-connector can be configured to be fixedly attached to a vertebra. The cross-connectors of the present invention can also include any combination of features described and/or illustrated herein, and the cross-connector is not limited to the illustrated embodiments.

As indicated above, the cross-connector in certain exemplary embodiments includes a central portion that extends between each connector member. The central portion can have variety of configurations. For example, it can be generally elongate to position the first and second connector members a distance apart from one another, or alternatively it can merely be formed from a direct connection between the first and second connector members. The central portion can also optionally have a fixed length, which can vary depending on the intended use, or alternatively the central portion can have an adjustable length. The central portion can also be angularly adjustable to allow the connector members to be positioned as desired. The adjustability of the cross-connector allows it to mate to parallel, non-parallel, diverging, and converging spinal rods that are implanted within a patient's spinal system.

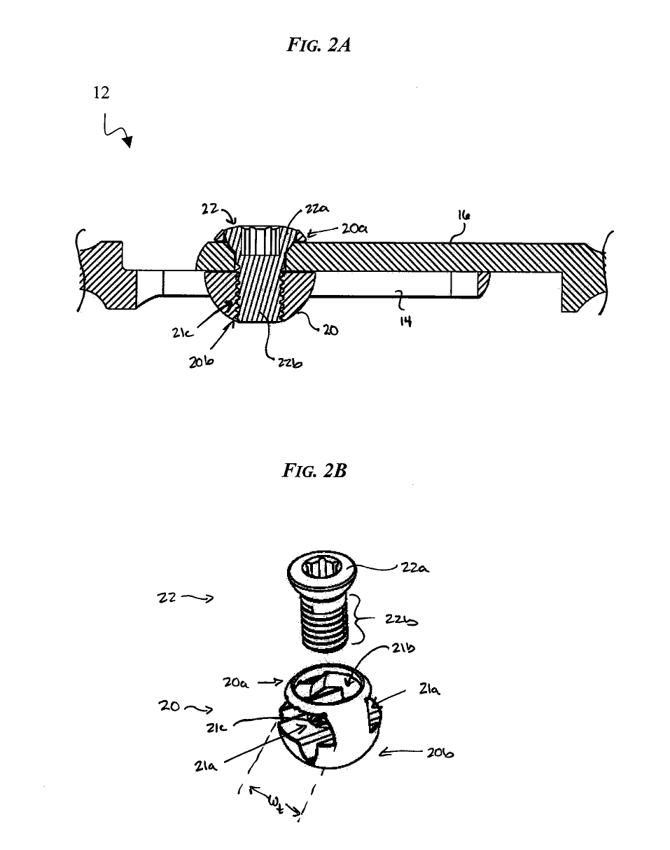

In the embodiment shown in FIGS. 1A-2A, the cross-connector 10 has a central portion 12 having an adjustable length, as well as an adjustable angle. As shown, the central portion 12 includes first and second generally elongate transverse members 14, 16 that extend along a longitudinal axis A of the cross-connector 10. The transverse members 14, 16 are slidably mated to one another by a central locking mechanism 18, which includes a clamp 20 for receiving a portion of each transverse member 14, 16, and a mating element 22 that extends through the clamp 20 and the transverse members 14, 16. The clamp 20 allows slidable movement of the transverse members 14, 16 therethrough, and the mating element 22 is effective to lock the clamp 20 and thereby lock the first and second transverse members 14, 16 in a fixed position with respect to one another.

The clamp 20 is shown in more detail in FIG. 2B, and it is in the form of a substantially hollow housing that defines a transverse pathway 21a extending therethrough along the longitudinal axis A of the cross-connector 10 for slidably receiving the first and second transverse members 14, 16. The clamp 20 also includes an enlarged proximal opening 21b formed in a proximal end 20a thereof, and a bore 21c formed in a distal end 20b thereof. The proximal opening 21b and bore 21c are adapted to receive a mating element that is effective to lock the clamp 20 and thereby lock the first and second transverse members 14, 16 in a fixed position with respect to one another. While virtually any mating element can be used, in an exemplary embodiment the mating element is a threaded member 22 having a head 22a that sits within the proximal opening 21b, and a threaded shank 22b that extends into the bore 21c, which is also preferably threaded for mating with the threaded shank 22b. When the threaded member 22 is mated to the clamp 20, the head 22a on the threaded member will engage the proximal end 20a of the clamp 20, and the threaded shank 22b will engage the distal end 20b of the clamp 20 such that rotation of the threaded member 22 will compress the clamp 20 by pulling the proximal and distal ends 20a, 20b of the clamp toward one another. In order to allow such rotation of the threaded member 22 with respect to the enlarged proximal opening 21b, the head 22a of the threaded member 22 is preferably non-eccentric, and more preferably it has a substantially circular or hemi-spherical shape.

The first and second transverse members 14, 16 can be mated to the clamp 20 by providing one or more openings formed in the transverse members 14, 16 for receiving the threaded member 22. As shown in FIGS. 1A-2A, the first transverse member 14 includes a slot 14c formed therein, and the second transverse member 16 includes an aperture 16c formed therein. When the threaded member 22 is disposed within the clamp 20, and it extends through the slot 14c in the first transverse member 14 and the aperture 16c in the second transverse member 16 to allow the first transverse member 14 to slide along the transverse pathway 21a extending through the clamp 20, thus allowing the length l₁₀ of the central portion 12 to be adjusted as desired. The threaded member 22 is preferably only loosely mated to the clamp 20 to allow such slidable movement. When the length l is adjusted as desired, the threaded member 22 can be tightened to compress the clamp 20 by pulling the proximal and distal ends 20a, 20b of the clamp 20 toward one another, thereby locking the first and second transverse members 14, 16 with respect to the clamp 20.

As previously noted, the transverse pathway 21a in the clamp 20 can also be adapted to allow angular adjustment of the first and second transverse members 14, 16 with respect to one another. In particular, as shown in FIG. 2B, the transverse pathway 21a can have a width wₜ that is greater than a width w, w (FIG. 1B) of one of both transverse members 14, 16. Since the second transverse member 16 is mated to the clamp 20 by an aperture 16a that receives a shank 22b of the threaded member 22, the enlarged width w of the pathway 21a allows the second transverse member 16 to be rotated about the shank 22b, thereby allowing the second transverse member 16 to be positioned at an angle with respect to the first transverse member 14. In an exemplary embodiment, one or both transverse members 14, 16 can be rotated about 20° in each direction with respect to the longitudinal axis A of the cross-connector 10, thus providing a combined angulation of about 40°. Once the desired angulation is obtained, the threaded member 22 can be securely fastened to the clamp 20 to lock the first and second transverse members 14, 16 at a fixed position with respect to one another.

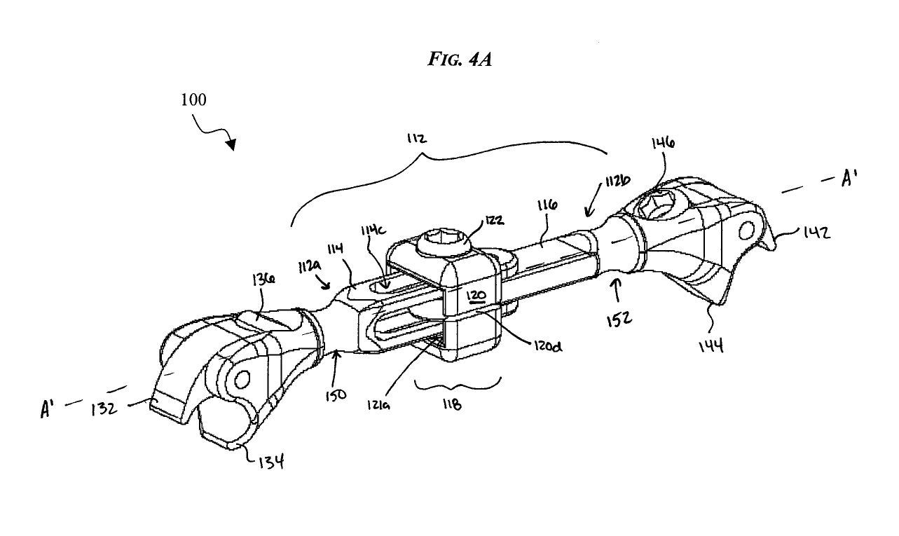

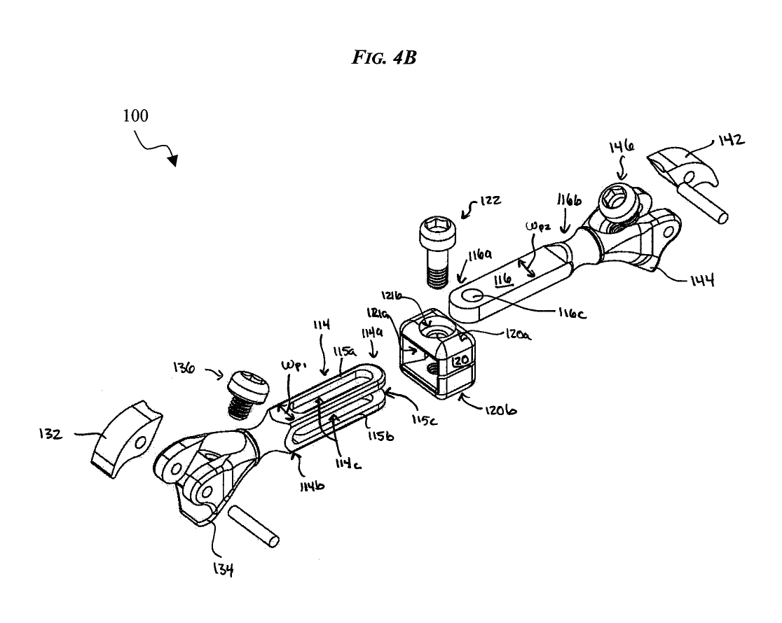

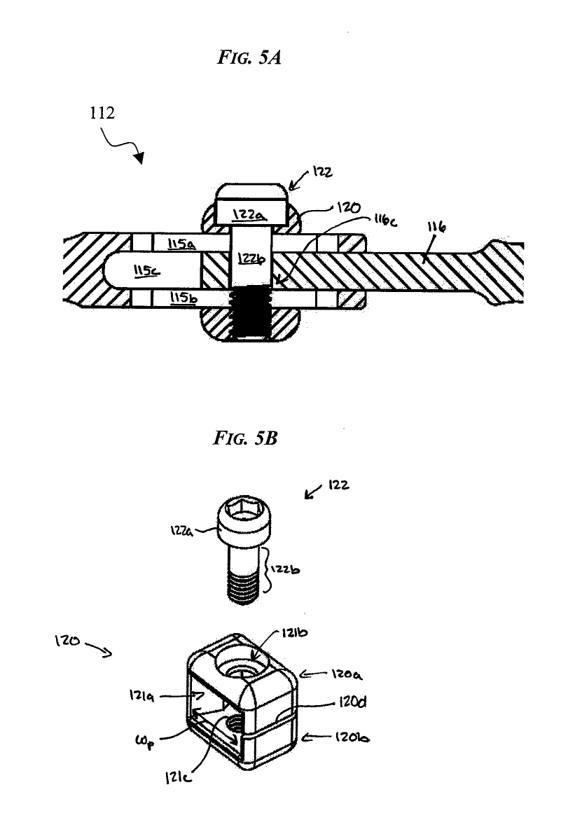

FIGS. 4A-5A illustrate another embodiment of a central portion 112 of a cross-connector 100 that has an adjustable length, i.e., that is telescoping, and that is angularly adjustable. As shown, the cross-connector 100 includes first and second transverse members 114, 116 that interlock to slidably mate to one another. The first transverse member 114 is in the form of male component having opposed arms 115a, 115b that define a longitudinally-extending receiving slot 115c therebetween for slidably receiving the second transverse member 116, which is in the form of a generally elongate female component. The transverse members 114, 116 also include openings formed therein for receiving a mating element, and in particular, the first transverse member 114 includes a longitudinally-extending slot 114c formed therein, and the second transverse members 116 includes an aperture 116c formed therein adjacent to a terminal end 116a thereof. As similarly described above with respect to cross-connector 10, the slot 114c and aperture 116c receive a mating element, e.g., threaded member 122, that extends therethrough, and that extends through a clamp 120 that forms part of the central locking mechanism 118.

The clamp 120 is shown in more detail in FIG. 5B and it has a substantially hollow, rectangular shape that defines a transverse pathway 121a extending therethrough for slidably receiving the first and second transverse members 114, 116. The pathway 121a preferably has a width wₚ that is greater than a width w, w of the transverse members 114, 116 to allow each of the first and second transverse members 114, 116 to be rotated and positioned at an angle with respect to one another. As previously described with respect to cross-connector 10, the first and/or second transverse members 114, 116 are preferably angularly adjustable about 20° in each direction with respect to a longitudinal axis A′ (FIG. 4A) of the cross-connector 100, for a combined angulation of about 40°. The clamp 120 also includes an enlarged opening 121b formed in a proximal end 120a thereof, and a threaded bore 121c (FIG. 4B) formed in a distal end 120b thereof. The opening 121b is adapted to seat the head 122a of the threaded member 122, and the threaded bore 121c in the distal end 120b of the clamp 120 mates with the threaded shank 122b on the threaded member 122. The clamp 120 also includes at least one longitudinal slit 120d formed therein that at least partially separates the proximal and distal portions 120a, 120b of the clamp 120 from one another. The slit 120d allows the threaded member 122 to compress the upper and lower portions 120a, 120b to lock the clamp 120 with respect to the first and second transverse members 114, 116, as described above with respect to cross-connector 10.

In yet another embodiment, the cross-connector can have a fixed length. By way of non-limiting example, FIG. 7 illustrates one such cross-connector 200 in which the central portion 212 is formed from a direction connection between the first and second connector members 230, 240. While not shown, the central portion 212 can optionally have an elongate configuration such that the first and second connector members 230, 240 are spaced a distance apart from one another, rather than directly connected to one another. The overall length l₂₀₀ of the cross-connector 200 can vary depending on the intended use, but in an exemplary embodiment the cross-connector 200 is provided as part of a kit that includes multiple cross-connectors, each having varying lengths l. In an exemplary embodiment, each cross-connector 200 has a length l that differs in increments of about 2 mm to 3 mm from one another, and that is in the range of about 18 mm to 30 mm for use in the thoracic spine.

In the embodiments described above with respect to FIGS. 1A-6B, the telescoping cross-connectors 10, 100 can also be provided as part of a kit having cross-connectors 10, 100 with a length l₁₀, l in the range of about 18 mm to 30 mm, differing in increments of about 2 mm to 3 mm. Cross-connectors having lengths in the range of 18 mm to 30 mm are particularly useful in the thoracic spine. The telescoping cross-connectors 10, 100 can also have lengths in the range of about 30 mm to 40 mm, for use in the lumbar spine, and lengths of about 40 mm to 118 mm for use in the cervical spine. Telescoping cross-connectors are particularly useful in the lumbar and cervical spine because most spinal fixation elements implanted therein are typically not parallel to one another. The telescoping configuration allows the necessary adjustments to be made for connecting non-parallel spinal fixation elements.

A person skilled in the art will appreciate that the central portion 12, 112, 212 of the cross-connector 10, 100, 200 can have a variety of other configurations, and that a variety of other techniques can be used to provide a cross-connector having an adjustable or telescoping length and/or having connector members that can be positioned at an angle with respect to one another.

In yet another embodiment, the cross-connector can include one or more bend zones formed thereon for allowing further angular adjustment of each connector member. While the location of the bend zone can vary, FIGS. 1A and 4A illustrate an exemplary embodiment of a cross-connector 10, 100 having a bend zone 50, 52, 150, 152 formed between the terminal end 12a, 12b, 112a, 112b of the central portion 12, 112 and each connector member 30, 40, 130, 140. The bend zone can also optionally be formed at a substantial mid-portion of the cross-connector, for example, as shown in FIG. 7, which illustrates cross-connector 200 having bend zone 250 formed between the opposed connector members 230, 240. Each bend zone 50, 52, 150, 152, 250 can be formed using a variety of techniques, but in the illustrated embodiments the bend zone 50, 52, 150, 152, 250 is formed by a decrease in diameter or thickness of the cross-connector 10, 100, 200. While the diameter or thickness at the bend zone 50, 52, 150, 152, 250 can vary, the bend zone 50, 52, 150, 152, 250 should allow the connector members 30, 40, 130, 140, 230, 240 to be angularly adjusted while still maintaining the structural integrity of the cross-connector 10, 100, 200. A person skilled in the art will appreciate that a variety of other techniques can be used to allow adjustable movement of each connector member 30, 40, 130, 140, 230, 240.

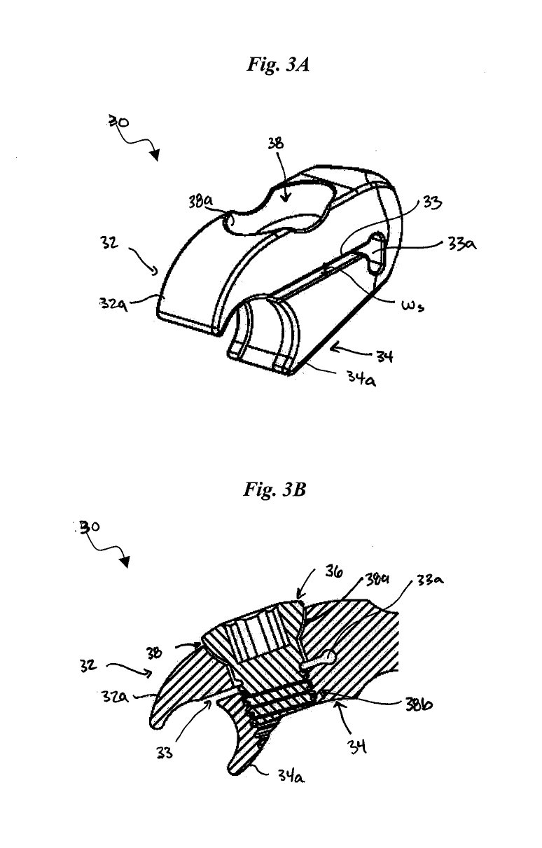

As previously stated, the cross-connector in certain exemplary embodiments also includes at least one connector member formed thereon, and each connector member can have a variety of configurations. In the embodiment illustrated in FIGS. 1A-2B, each connector member 30, 40 includes opposed first and second jaws 32, 34, 42, 44 formed thereon and fixedly attached to one another. For reference purposes, only one of the connector members, e.g., connector member 30, will be discussed, and it is shown in more detail in FIGS. 3A-3B. As shown, the first and second jaws 32, 34 have a generally elongate shape and they preferably include a terminal end 32a, 34a that is adapted to engage a fixation element therebetween. In the illustrated embodiment, the terminal ends 32a, 34a of the jaws 32, 34 define a substantially C-shaped recess for receiving and engaging a spinal fixation rod. The terminal end 32a, 34a of the jaws 32, 34 can also optionally include one or more gripping features formed thereon to facilitate grasping of a spinal fixation element. By way of non-limiting example, the gripping surface of each jaw 32, 34 can include a series of flats (not shown) to prevent slippage, and more particularly, the flats can be configured such that the jaws 32, 34, when viewed together, have a substantially octagonal configuration.

While a portion of the jaws 32, 34 are fixedly attached to one another, a slot 33 can extend between the jaws 32, 34 to at least partially separate the jaws 32, 34 to allow the terminal ends 32a, 34a of the jaws to move with respect to one another. In particular, the slot 33 preferably has a width w that is sufficient to allow the first and second jaws 32, 34 to be moved between an open position, as shown in FIG. 3A, and a closed position, in which the width w of the slot 33 is decreased and the jaws 32, 34 are pulled toward one another to engage a spinal fixation element positioned therebetween. In an exemplary embodiment, the slot 33 extends through a substantial portion of the connector member 30, as shown, and it includes an enlarged portion 33a formed at an end thereof to facilitate movement of the jaws 32, 34.

In order to move the first and second jaws 32, 34 toward one another, the connector member 30 also includes a locking mechanism that is adapted to come into contact with the first and second jaws 32, 34 to pull one or both jaws 32, 34 toward one another. While virtually any locking mechanism can be used, in an exemplary embodiment the locking mechanism is a threaded member 36 having a head 36a and a threaded shank 36b, as shown in FIGS. 3A-3B. The connector member 30 includes a bore 38 formed therein and extending through each of the first and second jaws 32, 34, as well as across the slot 33 formed between the jaws 32, 34. The bore 38 includes a proximal recess 38a formed in the first jaw 32 that is adapted to seat the head 36a of the threaded member 36, and a threaded distal portion 38b formed in the second jaw 34 that is adapted to mate to the threaded shank 36b on the threaded member 36. The threaded distal portion 38b is preferably only formed in the second jaw 34. In use, the threaded member 36 is effective to pull the first jaw 32 toward the second jaw 34 when the threaded member 36 is fastened within the bore 38, thereby decreasing the width w_{S} of the slot 33 and allowing the jaws 32, 34 to engage a spinal fixation element disposed therebetween.

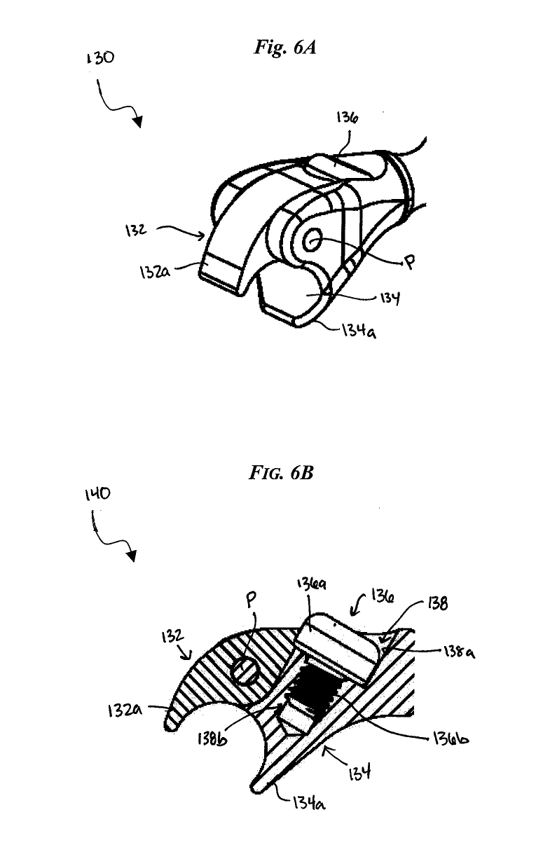

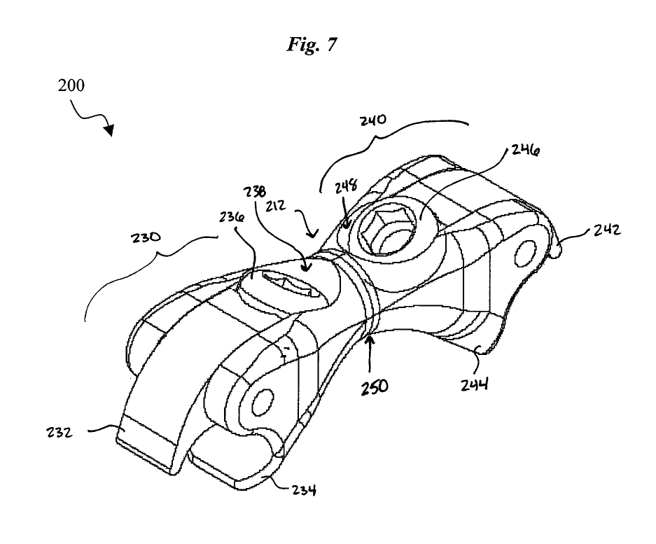

FIGS. 4A-4B illustrate another embodiment of a connector member in accordance with the present invention in which the jaws are pivotally mated to one another. For reference purposes, only one connector member, e.g., connector member 130, is discussed and it is shown in more detail in FIGS. 6A-6B. In this embodiment, rather than including a slot formed between the jaws for allowing movement of the jaws, as described above with respect to FIGS. 3A-3B, at least one of the jaws, e.g., the first jaw 132, is separate from, but pivotally mated to the other jaw, e.g., the second jaw 134. In particular, the second jaw 134, which is integrally formed with the connector member 100, is in the form of a substantially hollow housing having a cavity 137 formed therein for receiving the first jaw 132. The first jaw 132 has a generally elongate, curved configuration that is adapted to at least partially fit within the cavity 137 in the second jaw 134. A pivot element, such as a pivot pin P, extends through the first and second jaws 132, 134 to pivotally mate the jaws 132, 134 to one another. The first and second jaws 132, 134 also include a terminal portion 132a, 134a that defines a substantially C-shaped recess therebetween for engaging a spinal fixation element, and in particular a spinal fixation rod.

In use, the jaws 132, 134 are movable between an open position, in which the jaws 132, 134 are spaced apart from one another to allow a spinal fixation element to be positioned therebetween, and a second position, in which the first jaw 132 is pivoted toward the second jaw 134 to engage the spinal fixation element. The jaws 132, 134 can be locked in the second position using a locking element. As shown in FIGS. 6A-6B, a bore 138 is formed in the second jaw 134 for receiving a locking element, such as a threaded member 136. The bore 138 is preferably non-expandable and includes an enlarged opening or proximal recess 138a for receiving a head 136a on the threaded member 136, and a distal threaded portion 138b for mating with a threaded shank 136b on the threaded member 136. The proximal recess 138a is positioned such that the head 136a of the threaded member 136, when disposed within the bore 138, will abut the first jaw 132 and force the jaw 132 to pivot from the first position to the second closed position, thereby locking the first jaw 132 in the second closed position.

The pivoting jaw configuration is also illustrated in cross-connector 200 shown in FIG. 7, which includes a first jaw 232, 242 that is pivotally mated to a second jaw 234, 244.

A locking mechanism, e.g., a threaded member 236, 246, is disposable within a bore 238, 248 formed in each connector member 230, 240 for moving the first jaw 232, 242 to the second closed position, and for locking the first and second jaws 232, 242 in a fixed position with respect to one another.

A person skilled in the art will appreciate that a variety of other techniques can be used to move the jaws on the connector member between a first, open position and a second, closed position, and to lock the jaws in a fixed position with respect to one another to engage a spinal fixation element therebetween.

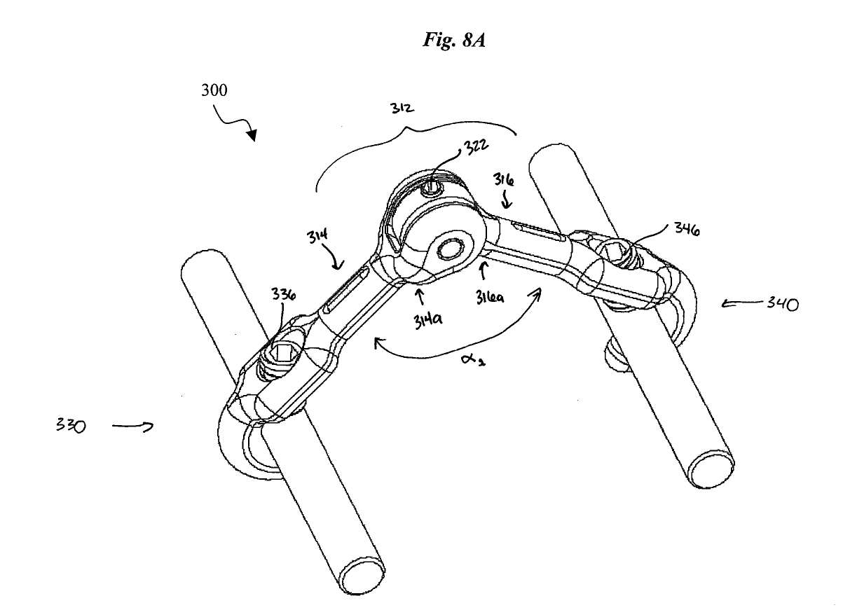

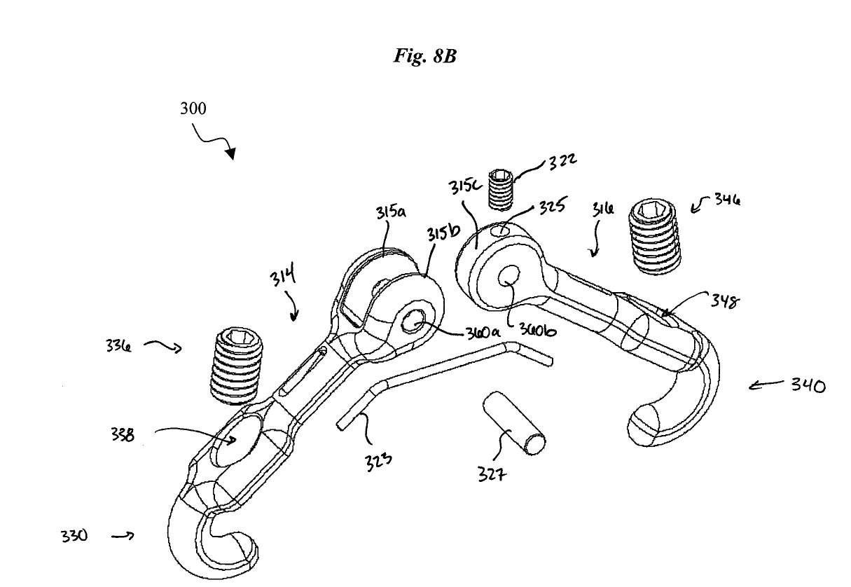

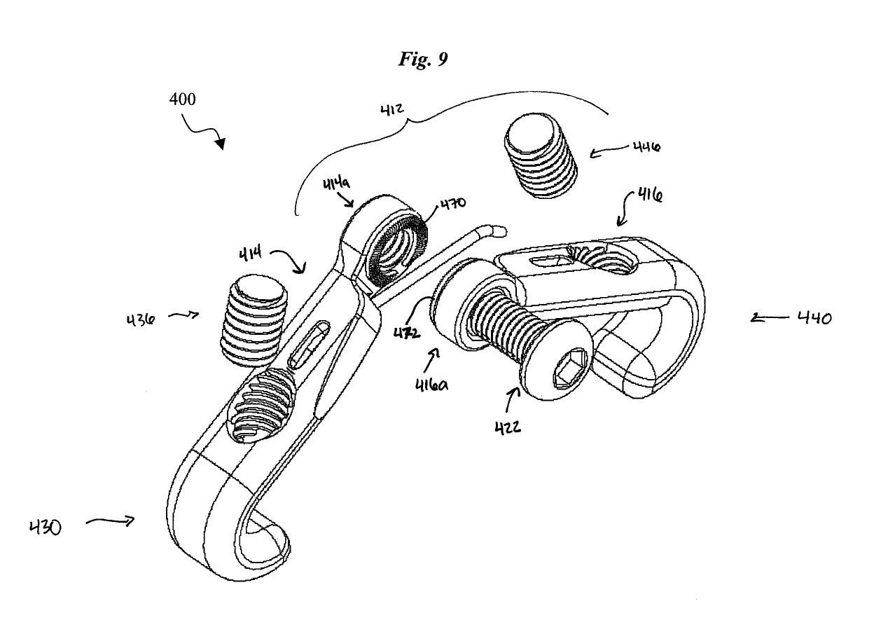

As shown in FIGS. 8A-9, the present invention also provides a cross-connector 300, 400 that utilizes a biasing element to facilitate placement and engagement of a spinal fixation element. The biasing element allows the cross-connector to be coupled to opposed spinal fixation elements disposed in a patient's spinal system prior to locking the cross-connector to each fixation element, thus providing a temporary, substantially secure connection.

Referring first to the embodiment illustrated in FIGS. 8A-8B, the cross-connector 300 includes a central portion 312 having first and second transverse members 314, 316 that are rotatably or pivotally mated to one another at a terminal end 314a , 316a thereof. In particular, the first transverse member 314 includes a central portion having opposed arms 315a, 315b that form a male component for receiving a corresponding female component 315c formed on the second transverse member 316. A pin 327 extends through a central aperture 360a, 360b formed in the opposed arms 315a, 315b and the female component 315c, and it fixedly mates to the arms 315a, 315b to allow rotation of the female component 315c with respect to the arms 315a, 315b. A locking mechanism, e.g., a threaded member 322, is provided for locking the first and second transverse members 314, 316 in a fixed position with respect to one another. The threaded member 322 is configured to extend into a second bore 325 formed in the second transverse member 316 that is in communication with the first bore 360b. This allows the threaded member 322 to engage the pin member 327 and prevent rotation of the first transverse member 314.

The cross-connector 300 also includes a biasing element, such as a spring 323 (FIG. 8B), that is adapted to biased the first and second transverse members 314, 316 to an open position, in which an angle α₁ between the first and second transverse members 314, 316 is maximized. The biasing element 323 preferably extends from the terminal ends 314a, 316a of the transverse members 314, 316 through at least a portion of each of the first and second transverse members 314, 316. In use, a force necessary to overcome the biasing force must be applied to the first and second transverse members 314, 316 to move the members 314, 316 to a second, closed position, in which the angle α₁ is decreased.

Each transverse member 314, 316 also includes a connector member 330, 340 formed thereon for engaging a spinal fixation element, such as a spinal rod 500, 502 as shown. While each connector member can have virtually any configuration, in this embodiment the connector members 330, 340 each have a substantially C-shaped jaw such that each transverse member 314, 316 and the connector member 330, 340 formed thereon is substantially J-shaped. Each connector member 330, 340 can also include a locking mechanism, e.g., a threaded member 336, 346, that is adapted to extend into a bore 338, 348 formed through each connector member 330, 340. In use, when a spinal fixation element, e.g., spinal rod 500, 502 is disposed within the C-shaped jaw of each connector member 330, 340, the threaded member 336, 346 can be threaded into the corresponding bore 338, 348 to engage the spinal rod 500, 502, thereby securely mating the connector member to the spinal rod 500, 502.

FIG. 9 illustrates a similar embodiment of a cross-connector 400 that utilizes an alternative locking mechanism for locking the position of the first and second transverse members 414, 416 with respect to one another. As shown, rather than including male and female components, each transverse member 414, 416 includes a central bore 460a, 460b extending through a terminal end 414a , 416a thereof. The bores 460a, 460b are adapted to be aligned when the terminal ends 414a , 416a are positioned adjacent to one another, and each bore 460a, 460b is threaded for receiving and mating with a threaded member 422. The transverse members 414, 416 also include teeth 470 and corresponding grooves 472 formed on the terminal ends 414a , 416a thereof such that, when the terminal ends 414a , 416a are positioned adjacent to one another, and the threaded member 422 is threaded through each bore 460a, 460b, the teeth and grooves prevent rotation of the first and second transverse members 414, 416 with respect to one another, thereby locking them in a fixed position.

One of ordinary skill in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

1. An implantable spinal cross-connector, comprising:
a central portion with at least one connector member formed on a terminal end thereof, the at least one connector member having
first and second opposed jaws, at least one of the jaws being selectively movable between a first, open position wherein the first and second jaws are positioned a distance apart from one another, and a second, closed position, wherein the first and second jaws are adapted to engage a spinal fixation element therebetween, and at least one of the jaws being integrally formed with the central portion, and
a locking mechanism having a shank that is receivable within a non-expandable bore formed in the connector member, the locking mechanism being adapted to come into contact with each of the first and second jaws to selectively lock at least one of the first and second jaws in a fixed position.

2. The implantable spinal cross-connector of claim 1, wherein the locking mechanism includes a non-eccentric head formed on a proximal end of the shaft.

3. The implantable spinal cross-connector of claim 2, wherein the non-expandable bore formed in the at least one connector member includes an enlarged proximal opening that is adapted to seat a non-eccentric head of the locking mechanism.

4. The implantable spinal cross-connector of claim 3, wherein the second jaw on the at least one connector member is pivotally mated to the first jaw, and wherein the non-eccentric head of the locking mechanism is effective to move the second jaw from the open position to the closed position when the head is disposed within the enlarged proximal opening of the non-expandable bore.

5. The implantable spinal cross-connector of claim 1, wherein the shank on the locking mechanism and the non-expandable bore include complementary threads formed thereon.

6. The implantable spinal cross-connector of claim 1, wherein the locking mechanism is adapted to pull the first and second jaws toward one another into the second, closed position when the locking mechanism is advanced into the non-expandable bore.

7. The implantable spinal cross-connector of claim 1, wherein the locking mechanism is adapted to push the second jaw toward the first jaw into the second, closed position when the locking mechanism is advanced into the non-expandable bore.

8. The implantable spinal cross-connector of claim 1, wherein the first and second jaws define a substantially C-shaped recess therebetween.

9. The implantable spinal cross-connector of claim 1, wherein the first and second jaws include a slot formed therebetween and adapted to allow movement of the first and second jaws between the first, open position and the second, closed position.

10. The implantable spinal cross-connector of claim 9, wherein the non-expandable bore extends through the first and second jaws across the slot such that the locking mechanism is effective to close the slot when the locking mechanism is advanced into the non-expandable bore, thereby moving the first and second jaws from the first, open position to the second, closed position.

11. The implantable spinal cross-connector of claim 10, wherein the non-expandable bore includes a non-threaded portion formed in the first jaw and a threaded portion formed in the second jaw, and wherein the shank of the locking mechanism includes a non-threaded proximal portion that is adapted to sit within the non-threaded portion of the non-expandable bore formed in the first jaw, and a threaded distal portion that is effective to mate with the threaded portion of the non-expandable bore formed in the second jaw.

12. The implantable spinal cross-connector of claim 11, wherein the non-threaded proximal portion of the shank of the locking mechanism further includes a head formed thereon that is receivable within an enlarged opening of the non-expandable bore formed in the first jaw.

13. The implantable spinal cross-connector of claim 1, wherein the first jaw is integrally formed with the at least one connector, and wherein the second jaw is independent from and pivotally mated to the first jaw.

14. The implantable spinal cross-connector of claim 13, further comprising a pivot pin extending through the first and second jaws to allow pivotal movement of the second jaw with respect to the first jaw.

15. The implantable spinal cross-connector of claim 13, wherein the locking mechanism includes a head formed on the shank that is receivable within an enlarged opening formed in the non-expandable bore, and wherein the head is adapted to pivotally move the second jaw from the first, open position to the second, closed position when the locking mechanism is disposed within the non-expandable bore and the head is disposed within the enlarged opening.

16. The implantable spinal cross-connector of claim 13, wherein the non-expandable bore is formed in the first jaw and it includes an enlarged opening formed therein for seating a head formed on the shank of the locking mechanism, the enlarged opening being formed adjacent to the second jaw such that the head of the locking mechanism is effective to pivot the second jaw into the second, closed position when the head is disposed within the enlarged opening.

17. The implantable spinal cross-connector of claim 16, wherein the non-expandable bore is threaded and the locking mechanism comprises a set screw having a threaded shank.

18. The implantable spinal cross-connector of claim 1, wherein the central portion comprises a substantially elongate member having an adjustable length.

19. The implantable spinal cross-connector of claim 18, wherein the substantially elongate member is formed from first and second transverse members that are slidably matable to one another.

20. The implantable spinal cross-connector of claim 19, wherein the first transverse member includes a female mating element, and the second transverse member includes a male mating element that is adapted to be received by the female mating element.

21. The implantable spinal cross-connector of claim 20, further comprising a central locking mechanism for locking the first and second transverse members at a fixed position with respect to one another.

22. The implantable spinal cross-connector of claim 19, wherein the first and second transverse members are angularly adjustable with respect to one another along a longitudinal axis of the spinal cross-connector.

23. The implantable spinal cross-connector of claim 22, wherein the first and second transverse members can be positioned at an angle of about 20° with respect to the longitudinal axis of the spinal cross-connector.

24. The implantable spinal cross-connector of claim 22, further comprising a central locking mechanism coupled to the first and second transverse members for allowing the first and second transverse members to be locked in a fixed position with respect to one another.

25. The implantable spinal cross-connector of claim 1, wherein the central portion includes first and second transverse members that are connected to one another by a central clamp that allows angular adjustment of the first and second transverse members with respect to one another along a longitudinal axis of the spinal cross-connector.

26. The implantable spinal cross-connector of claim 25, further comprising a central locking mechanism formed in the central clamp for locking the first and second transverse members in a fixed position with respect to one another.

27. The implantable spinal cross-connector of claim 26, wherein the central locking mechanism extends through the central clamp and each of the first and second transverse members, and wherein the locking mechanism is adapted to engage and close the central clamp, thereby locking the first and second transverse members therebetween.

28. The implantable spinal cross-connector of claim 1, wherein the at least one connector member is angularly adjustable with respect to the central portion.

29. The implantable spinal cross-connector of claim 28, wherein the at least one connector member includes a bend zone formed between the connector member and the central portion to allow angular movement of the connector member with respect to the central portion.

30. The implantable spinal cross-connector of claim 1, further comprising first and second connector members formed on opposed terminal ends of the central portion, and wherein the central portion includes a bend zone formed at a substantial mid-point thereof for allowing angular movement of each connector member with respect to the central portion.

31. The implantable spinal cross-connector of claim 1, wherein the first and second jaws each include a clamping surface formed thereon that is adapted to seat a spinal rod therebetween.

32. The implantable spinal cross-connector of claim 31, wherein the clamping surface of at least one of the first and second jaws includes at least one surface feature formed thereon to facilitate engagement of a rod between the first and second jaws.

33. The implantable spinal cross-connector of claim 32, wherein the surface feature comprises a series of ridges formed on the clamping surface.

34. The implantable spinal cross-connector of claim 1, wherein the central portion comprises first and second transverse members that are movable between an open position, in which the first and second transverse members are substantially longitudinally aligned with one another, and a second position, in which the first and second transverse members are positioned at an angle with respect to one another.

35. The implantable spinal cross-connector of claim 34, wherein the first and second members are biased to the second position.

36. The implantable spinal cross-connector of claim 1, wherein the spinal fixation element comprises a spinal rod.

37. An implantable spinal cross-connector, comprising:
at least one connector member having first and second opposed jaws that are biased to an open position, in which at least a portion of the first and second jaws are spaced apart from one another; and
a locking mechanism effective to engage at least one of the first and second jaws to move the jaws toward one another into a closed position, in which the jaws are effective to engage a spinal fixation element therebetween.

38. The implantable spinal cross-connector of claim 37, further comprising a bore formed in the first and second opposed jaws for receiving the locking mechanism.

39. The implantable spinal cross-connector of claim 38, wherein the first and second opposed jaws are at least partially separated by an elongate slot, and wherein the bore extends across the elongate slot.

40. The implantable spinal cross-connector of claim 39, wherein the locking mechanism comprises a threaded member, and wherein a portion of the bore formed in the second jaw is threaded to mate with the threaded member such that the locking mechanism is effective to move at least one of the first and second jaws toward one another to lock the jaws in the closed position.

41. A spinal rod and connector system, comprising:
at least one spinal rod;
a spinal cross-connector having
at least one connector member formed thereon and including first and second opposed jaws that are movable between an open position and a closed position in which the jaws are adapted to engage said spinal rod, at least one of the first and second jaws being integrally formed with the connector member, and
a locking mechanism having a head and a shank that are receivable with a bore extending through at least one of the first and second jaws, the bore having a proximal, head-receiving portion for seating the head of the locking mechanism, and a distal, shank-engaging portion for mating with the shank of the locking mechanism such that the locking mechanism is effective to lock the first and second jaws in the closed position.

42. The spinal rod and connector system of claim 41, wherein the second jaw on the at least one connector member is pivotally mated to the first jaw, and wherein the head of the locking mechanism is effective to move the second jaw from the open position to the closed position when the head is disposed within the proximal, head-receiving portion of the bore.

43. The spinal rod and connector system of claim 41, wherein the shank on the locking mechanism and the distal, shank-engaging portion of the bore include complementary threads formed thereon.

44. The spinal rod and connector system of claim 41, wherein the locking mechanism is adapted to pull the first and second jaws toward one another into the closed position when the locking mechanism is advanced into the bore.

45. The spinal rod and connector system of claim 41, wherein the locking mechanism is adapted to push the second jaw toward the first jaw into the closed position when the locking mechanism is advanced into the non-expandable bore.

46. The spinal rod and connector system of claim 41, wherein the first and second jaws include a slot formed therebetween, and wherein the bore extends through the first and second jaws across the slot such that the locking mechanism is effective to close the slot when the locking mechanism is advanced into the bore, thereby moving the first and second jaws from the open position to the closed position.

47. The spinal rod and connector system of claim 41, wherein the first jaw is integrally formed with the at least one connector, and wherein the second jaw is independent from and pivotally mated to the first jaw.

48. The spinal rod and connector system of claim 47, wherein the head on the locking mechanism is adapted to pivotally move the second jaw from the open position to the closed position when the locking mechanism is disposed within the bore.

49. The spinal rod and connector system of claim 41, further comprising first and second connector members that are coupled to one another by a central portion.

50. The spinal rod and connector system of claim 49, further comprising at least one bend zone formed in the central portion to allow the connector members to be positioned at an angle with respect to one another.
